(19) United States
(12) Patent Application Publication
(10) Pub. No.: US 2014/0041417 A1
(43) Pub. Date: Feb. 13, 2014

(54) DOUBLE-RASCHEL-KNITTED TUBE FOR ARTIFICIAL BLOOD VESSELS AND PROCESS FOR PRODUCING SAME

(71) Applicants: Yoshihide Takagi, Fukui-shi (JP); Nobuyuki Yamauchi, Kameoka-shi (JP); Shouichi Tsubota, Sakai-shi (JP)

(72) Inventors: Yoshihide Takagi, Fukui-shi (JP); Nobuyuki Yamauchi, Kameoka-shi (JP); Shouichi Tsubota, Sakai-shi (JP)

(21) Appl. No.: 14/000,044

(22) PCT Filed: Feb. 14, 2012
(86) PCT No.: PCT/JP2012/000954

§ 371 Date: Oct. 17, 2013

(30) Foreign Application Priority Data
Feb. 18, 2011 (JP) 2011-032982

(51) Int. Cl. A61L 27/22; A61F 2/06

(52) U.S. Cl. CPC A61L 27/225; A61F 2/06
USPC 66/202; 28/142; 28/168

(57) ABSTRACT

A method of manufacturing a double-raschel knitted fabric tube W for artificial blood vessels is provided, which is configured of N cocoon filaments each formed of paired core parts formed of fibroin and a skin part surrounding the core parts and formed of sericin, separating the paired core parts with the sericin partially removed and partially left to form 2N plies of the fibroin with a skin core structure to obtain a sericin-coated silk thread configured of the 2N plies of fibroin having the skin core structure, then subjecting the sericin-coated silk thread to a first twist to produce a first-twist silk thread, further combining a plurality of the first-twist silk threads together and subjecting the combined first-twist silk threads to a second twist to produce an organzine silk thread, and then further knitting a tube shape by using the organzine silk thread T as a knitting thread Y.

### TECHNICAL FIELD

The present invention relates to double-raschel-knitted tube for artificial blood vessels made of tube-shaped double-raschel knitted fabric knitted by a double-raschel loom and, in detail, to a double-raschel-knitted tube for artificial blood vessels knitted by using specially-processed silk threads and a method of manufacturing the tube.

### BACKGROUND ART

Regarding manufacture of artificial blood vessels, various suggestions have been made.

Among these suggestions, an example of means to form a tube-shaped artificial blood vessel is such that a tube shape is knitted, weaved, braided or the like by a knitting machine, a weaving machine, a braiding machine or the like, respectively. In another example, a fabric-like material is formed by sewing into a tube shape, which is used as an artificial blood vessel. In a further example, a synthetic high polymer, a natural high polymer or the like is molded directly into a tube shape.

And, in the case of a knitted, weaved, or braided product, polyester threads are mainstream as a material. An example of the synthetic high polymer is polytetrafluoroethylene (PTFE), and an example of the natural high polymer is chitosan.

The artificial blood vessel formed by the means described above is used, for example, temporarily as a blood flow path at the time of operation or the like, or for a long time inside the body as an alternative blood vessel in place of a blood vessel with a lesion.

Among these, the artificial blood vessel temporarily used at the time of operation or the like has been often used as a completed product with certain functionality.

Also, the artificial blood vessel as an alternative blood vessel in place of a blood vessel with a lesion is also used as a product usable inside the body for a certain period of time.

And, as materials relatively satisfying functional requirements for these artificial blood vessels, knitted products, in particular, warp-knitted fabric products, formed of tube-shaped polyester threads have been mainstream.

For example, PTL 1 suggests an artificial blood vessel formed of a warp-knitted fabric with a structure having cut ends less prone to raveling out. Also, PTL 2 suggests a tube-shaped double-raschel fabric using, as a material, a composite fiber with an elasticity similar to that of living blood vessels.

Furthermore, PTL 3 suggests an artificial blood vessel structured of an artificial blood vessel body of a double-raschel fabric using an elastic material similar to living blood vessels and an spiral-shaped elastic-material reinforcing part for reinforcing the artificial blood vessel body.

Still further, PTL 4 suggests an artificial blood vessel with its tube wall made of a porous high polymer compound.

As can be seen in these patent literatures described above, the artificial blood vessels include functions such as biocompatibility indicating the ability of the living body to accept and suturability with living blood vessels and, furthermore, structural contrivances resistant to compression and bending have been made.

For example, regarding the structural contrivances, means have been developed to provide a bellow shape to or mounting a ring-shaped reinforcing material on an artificial blood vessel. As for suturability, when an end hole of an artificial blood vessel and a living blood vessel are sutured together, some means is taken to prevent the end hole of the artificial blood vessel from being raveled out.

And, means for promoting fixing of endothelial cells onto artificial blood vessels and means for providing antithrombogenicity, and the like have been suggested to improve a patency rate.

Among these, as an artificial blood vessel not based on polyester threads, PTL 5 suggests an artificial blood vessel with a braded structure using raw silk, and PTL 6 suggests an artificial blood vessel based on a tube-shaped warp-knitted fabric or the like using silk threads.

Also, PTL 7 suggests an artificial blood vessel formed of a bioabsorbable material and degraded and absorbed into the living body after a self organization is reproduced.

### CITATION LIST

### Patent Literatures

PTL 1: Japanese Patent Application Laid-Open No. 5-161664

PTL 2: Japanese Patent Application Laid-Open No. 5-161708

PTL 3: Japanese Patent Application Laid-Open No. 5-337143

PTL 4: Japanese Patent Application Laid-Open No. 5-76588

PTL 5: Japanese Patent Application Laid-Open No. 2004-173772

PTL 6: Japanese Patent Application Laid-Open No. 2009-279214

PTL 7: Japanese Patent Application Laid-Open No. 2001-78750

### SUMMARY OF INVENTION

### Technical Problem

As described above, many suggestions and improvements have been provided to the artificial blood vessels, which have been used by making the most of their features.

However, there are still many problems to be solved as to whether the present artificial blood vessels are functioning similarly to the living blood vessels over a long period of time in the living body.

That is, the artificial blood vessel for use as an alternative blood vessel for a blood vessel with a lesion is required to have a performance capable of, after becoming a living body, functioning as a blood vessel over a long period of time. Examples of functional conditions required for becoming a living body include quick formation of endothelial cells with favorable biocompatibility with blood and internal tissues, reliable fixing of the endothelial cells, and antithrombogenicity. Ideally, what is required is a high patency rate; high resistance to degradation of blood, internal tissues, and the like in an internal environment; a strength resistible to blood pressure, heart pulsation, and the like; and, finally, complete formation of living blood vessels after the artificial blood vessels are absorbed to the inside of the body and lost.

For these requests, although not rejected by the living body, artificial blood vessels using polyester threads in the mainstream have problems such that fixing of internal tissues is uncertain and a thrombus tends to be formed, and are not perfect as artificial blood vessels.

In particular, although products usable as artificial blood vessels with a large diameter have been developed, artificial blood vessels with a small diameter (tube outer diameter) smaller than a 5-mm level have obstructive and other problems. Under present circumstances, products sufficiently resistible for use have not been developed yet.

To solve these problems, a product using a porous elastomer has also been suggested, but has problems in strength and internal degradation, and therefore cannot be said as perfect.

Regarding these problems and background, PTL 5 and PTL 6 mentioned above suggest artificial blood vessels using silk (raw silk), and PTL 7 suggest ideal artificial blood vessels that are dissolved and absorbed after functioning as artificial blood vessels for a certain period of time to cause formation of a self organization.

Among these, the artificial blood vessels with a braided structure using raw silk in PTL 5 have some advantage. However, as a feature of the braided structure, these blood vessels tend to extend when a tensile force is exerted in a longitudinal direction and, as a result, the tube diameter disadvantageously becomes narrow.

If adhesive means is taken to prevent this, a problem such as hardening is disadvantageously derived.

Moreover, while the type having a dissolvable and absorbable function described in PTL 7 is ideal, there are problems such that adjustment between a period in which a self organization is formed and a period in which the blood vessels are dissolved and absorbed cannot be ensured. Therefore, this type cannot achieve a sufficient function as artificial blood vessels.

By contrast, the suggestion described in PTL 6 attracts attention as a suggestion achieving both of an advantage of a tube-shaped knitted fabric using polyester threads currently in the main stream and an advantage of silk with proven biocompatibility as used for medical ligatures.

It is a great advantage to use silk threads, which has proven biocompatibility, as in PTL 6, and a suggestion of forming a tube-shaped warp-knitted fabric with the silk threads attracts attention as a new suggestion compared with conventional suggestions.

Cocoon filaments discharged from silkworms are natural filament fiber. A core part made of two components called fibroin and each having an approximately triangular sectional shape is coated with a colloid protein called sericin serving as a skin part.

And, as well known, fibroin and sericin are both protein with amino acid such as glycine and alanine as a basic composition and have biocompatibility.

Meanwhile, although it seems advantageous to use silk threads for artificial blood vessels, a big problem is that no technical measure for forming a tube-shaped double-raschel fabric by using silk threads has been suggested at all.

That is, under present circumstances, no technical measure for efficiently forming a double-raschel-knitted tube for artificial blood vessels, in particular, a small-diameter tube-shaped double-raschel-knitted tube for artificial blood vessels, by using silk threads as knitting threads has not been established at all.

This is because a mutual contact relation between a guide and a needle of a double-raschel loom and silk threads as knitting threads has not been sufficiently studied.

As for knitting with silk threads by a warp knitting machine, silk threads have been conventionally used for knitting for clothing by a tricot machine, which is a type of warp knitting machines. In the mechanism of the tricot machine, excessive tension is not exerted on the knitting threads, and therefore knitting with silk threads can be performed.

However, in the mechanism of the double-raschel loom, which is also a type of warp knitting machines and is for knitting a double-raschel fabric, considerable tension load is exerted on the knitting threads. In the case of fine silk threads, thread breakage and fluffing disadvantageously occur to make efficient knitting impossible.

The present invention was made with these circumstances as described above as backgrounds.

That is, an object of the present invention is to provide a method of manufacturing a double-raschel-knitted tube for artificial blood vessels by efficient knitting with minimized occurrence of thread breakage and fluffing.

Furthermore, the present invention is to provide the double-raschel-knitted tube for artificial blood vessels obtained by the manufacturing method.

### Solution to Problems

To solve the problems described above, the inventors have found as a result of diligent studies that sericin-coated silk threads having the residual sericin is used and the sericin-coated silk threads is subjected to a special twist process to bring knitting in a suitable state.

Based on these findings, the inventors have developed manufacture of a tube-shaped double-raschel-knitted tube for artificial blood vessels in a tube shape.

That is, the present invention resides in (1) a method of manufacturing a double-raschel-knitted tube for artificial blood vessels, the method comprising: refining raw silk configured of N cocoon filaments each formed of paired core parts formed of fibroin and a skin part surrounding the core parts and formed of sericin; separating the paired core parts with the sericin partially removed and partially left to form 2N plies of fibroin with a skin core structure to obtain a sericin-coated silk thread configured of the 2N plies of fibroin having the skin core structure; then subjecting the sericin-coated silk thread to a first twist to produce a first-twist silk thread; further combining a plurality of the first-twist silk threads together and subjecting the combined first-twist silk threads to a second twist to produce an organzine silk thread; and then further knitting a tube shape by using the organzine silk thread as a knitting thread.

That is, the present invention resides in (2) the method of manufacturing the double-raschel-knitted tube for artificial blood vessels according to 1 mentioned above, wherein the organzine silk thread has a number of twists for the second twist in a direction in reverse to a first twist direction of the first-twist threads, the number of twists for the first twist being smaller than a number of twists for the first twist.

That is, the present invention resides in (3) the method of manufacturing the double-raschel-knitted tube for artificial blood vessels according to 1 or 2 mentioned above, wherein the residual sericin is 20% to 40% of a total sericin weight contained in one thread of the raw silk.

That is, the present invention resides in (4) the method of manufacturing the double-raschel-knitted tube for artificial blood vessels according to any one of 1 to 3 mentioned above, wherein the first-twist silk threads are two threads.

That is, the present invention resides in (5) the method of manufacturing the double-raschel-knitted tube for artificial blood vessels according to any one of 1 to 4 mentioned above, wherein the organzine silk thread is used to knit the tube shape with a double Denbigh tissue.

That is, the present invention resides in (6) the method of manufacturing the double-raschel-knitted tube for artificial blood vessels according to any one of 1 to 4 mentioned above, wherein the organzine silk thread is used to knit the tube shape with a reverse half tissue.

That is, the present invention resides in (7) the method of manufacturing the double-raschel-knitted tube for artificial blood vessels according to any one of 1 to 6 mentioned above, wherein the first twist is 900 t/m to 1200 t/m, the second twist is 700 t/m to 1000 t/m, a finishing course density is 40 c/inch to 50 c/inch, and a finish wale density of 50 w/inch to 70 w/inch.

That is, the present invention resides in (8) the method of manufacturing the double-raschel-knitted tube for artificial blood vessels according to any one of 1 to 7 mentioned above, wherein a tube wall thickness is 0.1 mm to 0.3 mm, and a tube outer diameter is 1 mm to 5 mm.

That is, the present invention resides in (9) the method of manufacturing the double-raschel-knitted tube for artificial blood vessels according to any one of 1 to 8 mentioned above, wherein an enzyme is used to refine the sericin.

That is, the present invention resides in (10) the method of manufacturing the double-raschel-knitted tube for artificial blood vessels according to any one of 1 to 9 mentioned above, wherein the residual sericin is removed after knitting.

That is, the present invention resides in (11) a method of manufacturing a double-raschel-knitted tube for artificial blood vessels obtained by subjecting a sericin-coated silk thread to a first twist to produce a first-twist silk thread, and further combining a plurality of the first-twist silk threads together and subjecting the combined first-twist silk threads to a second twist to produce an organzine silk thread and knitting a tube shape by using the organzine silk thread as a knitting thread, wherein the sericin-coated silk thread is configured of fibroin with a skin core structure, the fibroin with the skin core structure being formed by refining raw silk configured of a cocoon filament formed of paired core parts formed of fibroin and a skin part coating the core parts and formed of sericin and separating the paired core parts with the sericin partially removed and partially left.

Note that a structure obtained by combining (1) to (11) mentioned above as appropriate can be adopted as long as the structure is along the object of the present invention.

### Advantageous Effects of Invention

According to the double-raschel-knitted tube for artificial blood vessels and the method of manufacturing the tube of the present invention, sericin-coated silk threads configured of fibroin with a skin core structure with partially residual sericin are used. Therefore, the guide and the needle make contact not with the core part but with sericin of the skin part, at the time of knitting, thereby preventing fibroin of the core part from being damaged.

Also, damage on the core part due to direct pressure between core parts can be solved.

In addition, sericin of the skin part serves as lubrication when a sericin-coated silk thread is guided to the needle. With this synergy effect, problems such as thread breakage and fluffing can be prevented to allow efficient knitting, thereby obtaining a high-quality double-raschel-knitted tube for artificial blood vessels.

Also, since a special twist process is performed on the sericin-coated silk threads, the sericin-coated silk threads become in a stable state without a running torque, thereby obtaining an appropriate convergence effect.

As a result, even if the thread is pressed due to the contact with the guide and the needle at the time of knitting, the section is prevented from being extremely flattened, thereby effectively reducing contact friction with the contact part becoming in a state of almost a point contact.

In addition, when a new needle loop exits an old needle loop in the knitting process, the contact part therebetween becomes in a state of almost a point contact, thereby reducing contact friction, achieving extremely smooth knitting, preventing problems such as thread breakage and fluffing, and easily achieving a stable high-quality double-raschel-knitted tube for artificial blood vessels having a small tube outer diameter without a kink or the like.

And, in the double-raschel-knitted tube for artificial blood vessels knitted by using sericin-coated silk threads with partially residual sericin, the residual sericin amount can be adjusted by postprocessing such as further removing the residual sericin as appropriate so that the residual sericin amount is at a predetermined level. By selecting a residual sericin amount according to the use purpose or use region, the range of use purpose can be advantageously widened to a great extent.

And, when knitting is performed by using sericin-coated silk threads as knitting threads for the double-raschel-knitted tube for artificial blood vessels, adopting a double Denbigh texture or a reverse half texture as a knitting texture, setting a finishing course density of 40 c/inch to 50 c/inch, and setting a finish wale density of 50 w/inch to 70 w/inch, a thin and dense knitted fabric is obtained, and it is possible to suppress the tube wall thickness of the double-raschel-knitted tube for artificial blood vessels to 0.1 mm to 0.3 mm and the tube outer diameter thereof to 1 mm to 5 mm (as a matter of course, 5 mm or larger is possible). This can be used as an alternative blood vessel for a peripheral blood vessel having a small diameter, which is conventionally impossible.

Also, blood leakage, which is problematic at the time of use as an artificial blood vessel, can be suppressed, and the function as an artificial blood vessel can be sufficiently obtained.

And, since the enzyme is used in refining raw silk, quantitative fine adjustment of the residual sericin can be made in a range of 20 percent to 40 percent of a total sericin weight contained in one raw silk thread. In addition, sericin-coated silk threads with an extremely uniform skin part of fibroin having a skin core structure can be formed and, as a result, a high-quality double-raschel-knitted tube for artificial blood vessels according to the use purpose can be obtained.

### BRIEF DESCRIPTION OF DRAWINGS

FIG. 1 is an actual schematic view (partially omitted) of a double-raschel knitted fabric tube W for artificial blood vessels.

FIG. 2 is a texture diagram of a double Denbigh texture B (an enlarged view of a texture portion P of FIG. 1, partially indicated by bold lines).

FIG. 3 is a texture diagram of a reverse half texture H (an enlarged view of the texture portion P of FIG. 1, partially indicated by bold lines).

FIG. 4 is a partial descriptive view depicting a positional relation of reeds (L1 to L6), a needle N, and knitting threads Y in a double-raschel loom R.

FIG. 5 is an actual schematic view (partially omitted) of a cocoon filament 1.

FIG. 6 is an actual schematic view (partially omitted) of raw silk 2.

FIG. 7 is an actual schematic view (partially omitted) of silk threads 3.

FIG. 8 is an actual schematic view (partially omitted) of fibroin 4 with a skin core structure.

FIG. 9 is an actual schematic view (partially omitted) depicting a twist direction of a second-twist silk thread T1.

FIG. 10 is an actual schematic view (partially omitted) depicting a twist direction of organzine silk threads T1.

FIG. 11 is a state descriptive diagram (partially omitted) depicting the state in which a needle loop N1 exits a needle loop N2 when the double-raschel knitted fabric tube W for artificial blood vessels is knitted.

FIG. 12 is a state descriptive diagram (partially omitted) depicting a contact state between the needle loop N1 and the needle loop N2.

### DESCRIPTION OF EMBODIMENTS

With reference to the drawings as required, preferable embodiments of the present invention are described in detail below.

### First Embodiment

In the present embodiment, a special process of removing sericin as appropriate is performed on raw silk, then a silk thread obtained thereby is subjected to a twist process and, furthermore, this silk thread is used to knit a tube shape by a double-raschel loom.

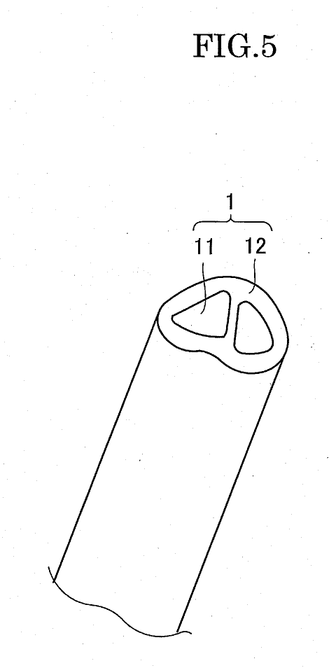

As depicted in an actual schematic view (partially omitted) of FIG. 5, a thread discharged from a silkworm is a cocoon filament 1. This cocoon filament has paired core parts 41 each having a triangle-shaped section and formed of fibroin 11 coated with sericin 12, which is a colloid protein serving as a skin part 42.

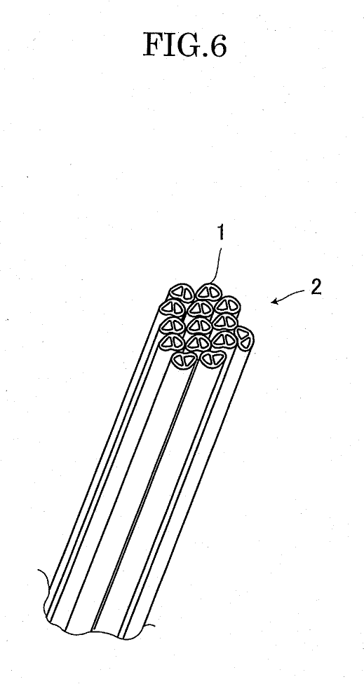

And, a plurality of these cocoon filaments 1 combined together are raw silk 2, as illustrated in an actual schematic view (partially omitted) of FIG. 6. In FIG. 6, fourteen cocoon filaments 1 are combined together to form the raw silk 2.

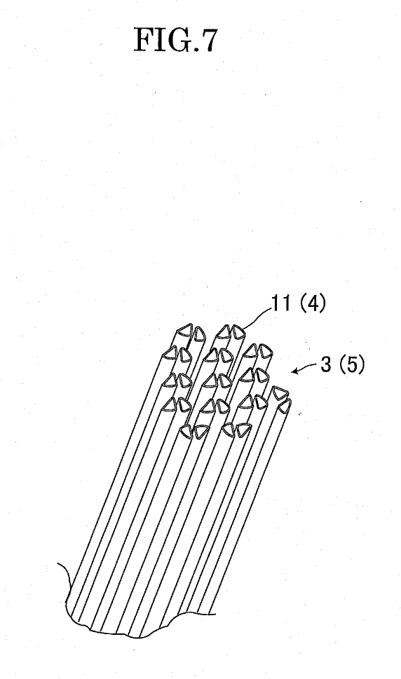

Since the raw silk 2 is formed by combining the cocoon filaments 1 together as they are, the sericin 12 is still attached. Threads obtained by removing the sericin 12 from the raw silk 2 and separating the raw silk into two plies of fibroin 11 are called silk threads 3, as depicted in an actual schematic view (partially omitted) of FIG. 7.

In FIG. 7, twenty-eight plies of fibroin 11 are depicted. And, a process of removing the sericin 12 from the raw silk 2 is called refinement.

As can be seen from the above description, when the raw silk 2 configured of N cocoon filaments 1 is refined and a predetermined amount of the sericin 12 is removed, the fibroin 11 of the paired core parts 41 is separated and, as a result, a silk thread 3 configured of 2N plies of fibroin 11 is obtained.

In the drawing example, fourteen cocoon filaments 1 depicted in FIG. 6 become twenty-eight (14×2) plies of fibroin.

In the present embodiment, the raw silk 2 is refined by using an enzyme, thereby removing 70% of a total sericin weight contained in one thread of the raw silk 2 and leaving 30% thereof, for example.

In this connection, the total sericin weight contained in one thread of the raw silk 2 is in a range of 20% to 30% of a total weight of one thread of the raw silk 2.

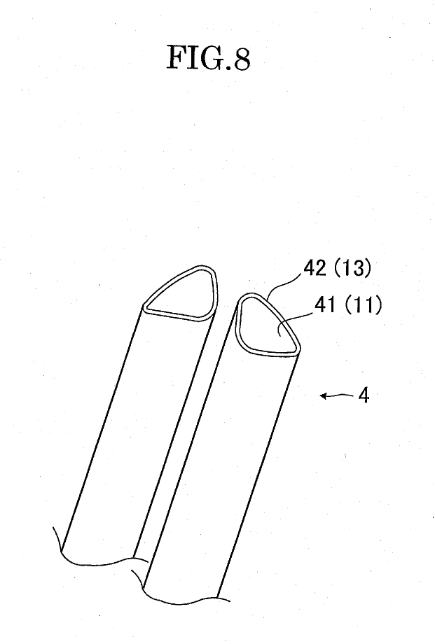

As a result, as depicted in an actual schematic view (partially omitted) of FIG. 8, the fibroin 11 is in a state of being coated with residual sericin 13 in a thinly residual state.

Here, as an enzyme for use in refinement, for example, protease (“Esperase” (product name); manufactured by Novozymes Japan Ltd.) is preferably adopted.

That is, the fibroin 4 with the skin core structure is formed in the state where the residual sericin 13 serves as the skin part 42 and the fibroin 11 of the core part 11 is thinly coated.

The silk thread 3 configured of the fibroin 4 with the skin core structure is referred herein as a sericin-coated silk thread 5.

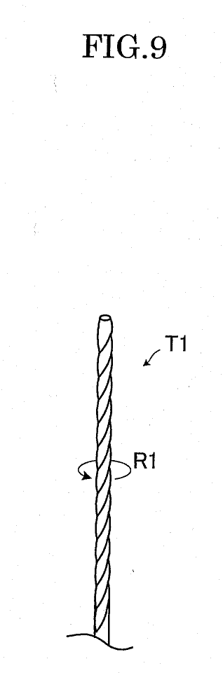

The state of the sericin-coated silk thread 5 is similar in external appearance to the state of the silk thread 3 depicted in FIG. 7 previously described, and therefore FIG. 9 is used as its actual schematic view.

Here, the amount of sericin to be left described above is preferably 20% to 40% in view of separation of the fibroin 11 of the core parts 41 and a coating effect, which will be described further below.

Also in the present embodiment, the raw silk 2 of 21 d (deniers) is used.

In this connection, raw silk of 14 d, 17 d, 21 d, and 31 d (deniers) are mainly adopted.

The raw silk 2 of 21 d is normally configured of fourteen to sixteen cocoon filaments 1. And, this raw silk 2 is refined by using the enzyme.

As described above, when N cocoon filaments 1 are refined, 2N plies of fibroin 11 are obtained. Therefore, twenty-eight to thirty-two plies of fibroin 11 of the raw silk 2 of 21 d are obtained and, as a result, the sericin-coated silk thread 5 configured of twenty-eight to thirty-two plies of fibroin 4 with the skin core structure.

In the refining process, a cleaning process and a drying process are provided to clean and remove dirt and needless sericin, and then mold or the like is prevented by drying.

Next, a two-step twist process is performed to provide a special twist state to thus obtained sericin-coated silk thread 5.

As depicted in an actual schematic view (partially omitted) of FIG. 9, for example, a first twist of 1100 t/m is first performed with a left twist (a left twist direction R1) to obtain a first-twist silk thread T1.

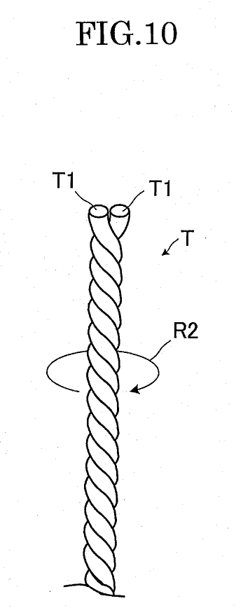

Then, as depicted in an actual schematic view (partially omitted) of FIG. 10, two first-twist silk threads T1 are combined together, and a second twist of 900 t/m is performed with a right twist (a right twist direction R2) this time to obtain an organzine silk thread T.

As a range of the twist process described above, 900 t/m to 1200 t/m is preferably adopted for the first twist, and 700 t/m to 1000 t/m is preferably adopted for the second twist.

By making the first twist direction and the second twist direction in reverse to each other and reducing the number of twists for the first twist so that it is less than the number of twists of the second twist, the organzine silk thread T in a stable state without a running torque can be obtained.

With this, thread breakage and fluffing can be prevented, and efficient knitting is performed.

Also, the knitted double-raschel-knitted tube for artificial blood vessels is of high quality without a kink.

Regarding how much the number of twists for the second twist is less than the number of twists for the first twist, the number of twists for the second twist is determined so that the most stable state without a running torque is achieved when two first-twist silk threads T1 are combined together to produce the organzine silk thread T.

The twist process described above can be summarized as follows. A plurality of sericin-coated silk threads 5 are subjected to a first twist in the same direction and with the same number of twists to produce the first-twist silk thread T1. A plurality of first-twist silk threads T1 are combined together and subjected to a second twist in a reverse direction and with the number of twists less than the number of twists for the first twist, thereby obtaining the organzine silk thread T.

Here, the plurality means herein that the number of the first-twist silk threads T1 is not necessarily required to be two and three or more may suffice according to the purpose.

By performing a special twist process on the sericin-coated silk thread 5 in this manner, the sericin-coated silk threads 5 become in a stable state without a running torque, thereby obtaining an appropriate convergence effect.

As a result, even if the thread is pressed due to the contact with the guide and the needle at the time of knitting, the section is prevented from being extremely flattened, thereby reducing contact friction with the contact part becoming in a state of almost a point contact (a twist effect).

Also, since the core parts 41 do not make direct contact with each other, the problems of fluffing and the like are solved, and smooth knitting is performed.

On the other hand, with the residual sericin 13 of the skin part coating the core parts 41, not the core parts 41 but the skin part 42 makes a contact with the guide and the needle at the time of knitting.

Also, since no direct pressure contact between the core parts 41, the core parts 41 are not damaged (a first coating effect)

And, the sericin of the skin part 42 plays a role of lubrication when the sericin-coated silk thread is guided to the needle (a second coating effect). Therefore, problems such as thread breakage and fluffing can be more effectively prevented.

These merits also apply at the time of forming a needle loop in knitting.

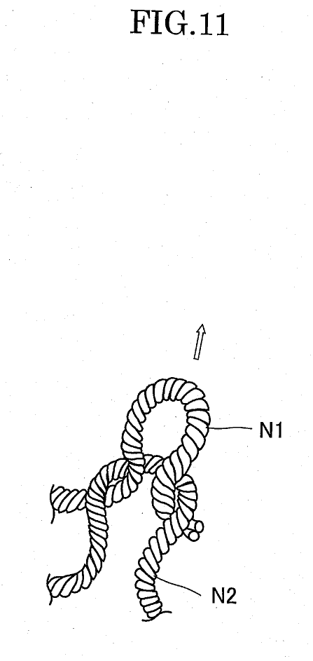

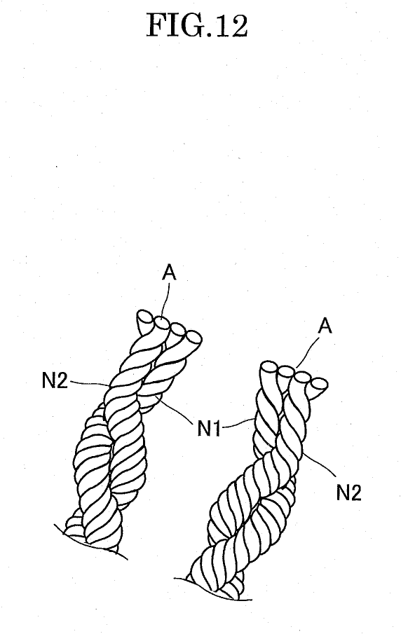

That is, as depicted in a state descriptive diagram (partially omitted, and an actual knitted fabric is densely knitted and gaps as in this drawing are much less) of FIG. 11, when a new needle loop N1 exits an old needle loop N2 in the knitting process, the needle loops N1 and N2 have a contact part A in a state of almost a point contact due to a special twist process, as depicted in a state descriptive diagram (partially omitted) of FIG. 12. With this, contact friction is reduced and, similarly to the above, together with the coating effect by the residual sericin 13 of the skin part 42, extremely smooth knitting is performed.

As described above, the problems such as thread breakage and fluffing are solved, and therefore a high-quality double-raschel knitted fabric tube W for artificial blood vessels with a small tube outer diameter can be easily provided.

By performing the twist process, no fluffing occurs and therefore the surface becomes smooth, which is effective in improving functionality as an artificial blood vessel, thereby obtaining a high-quality double-raschel knitted fabric tube W for artificial blood vessels.

Meanwhile, the residual sericin 13 in the skin part 42 may be removed according to the use purpose of the double-raschel knitted fabric tube W for artificial blood vessels after knitting.

That is, there are the case of leaving the residual sericin as it is, the case of further removing the residual sericin by one step, or the case of completely removing the residual sericin.

As such, options for adjusting the sericin residual amount can be provided, the use purpose range of the double-raschel knitted fabric tube W for artificial blood vessels is effectively enlarged.

Next, knitting of the double-raschel-knitted tube for artificial blood vessels is described.

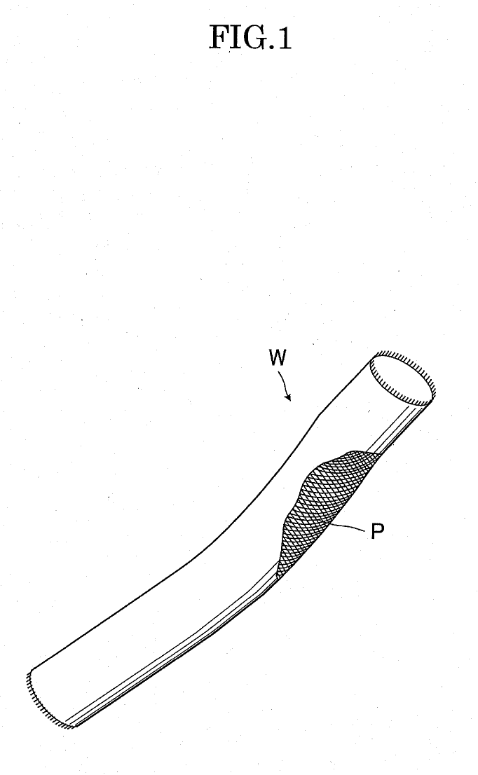

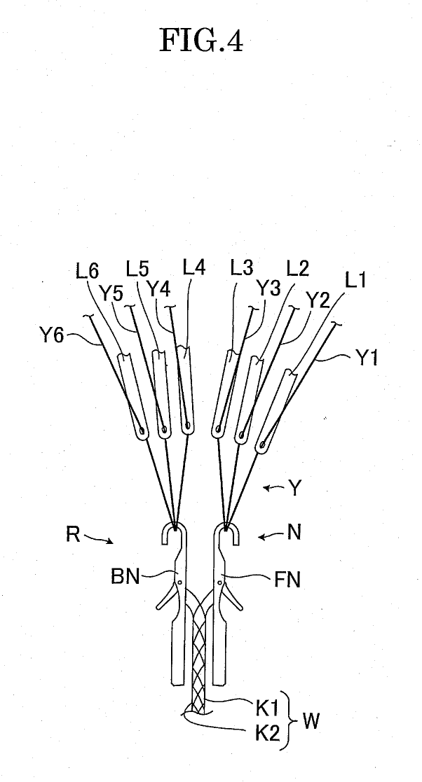

As a partial descriptive diagram of FIG. 4, the organzine silk threads T are used as knitting threads Y (Y1 to Y6) and, with a double-raschel loom R with six reeds (twenty-eight gauges), the tube-shaped double-raschel knitted fabric tube W for artificial blood vessels depicted in FIG. 1 is knitted.

Here, the gauge indicates the number of needles N that are present in one inch (2.54 cm).

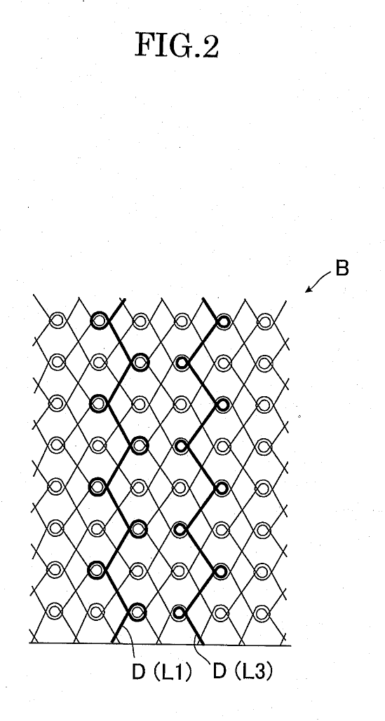

As a knitted tissue, a double Denbigh tissue B (1-0/1-2 is knitted by the reed L1 and 1-2/1-0 is knitted by the reed L3) depicted in FIG. 2 (which is an enlarged view of a portion P in FIG. 1, and parts are indicated by bold lines for ease of understanding) is adopted.

The double Denbigh tissue B is obtained by simultaneously knitting two Denbigh tissues D.

In FIG. 4, the knitting threads Y1 and Y3 are controlled by the reeds L1 and L3, and a front knitted fabric K1 of the double Denbigh tissue B is knitted by a front needle FN.

And, the knitting threads Y4 and Y6 are controlled by the reeds L4 and L6, and a back knitted fabric K2 of the double Denbigh tissue B is knitted by a back needle BN.

Thus knitted fabrics K1 and K2 of the double Denbigh tissue B of two tissues are coupled together with the knitting threads Y2 and Y5 controlled by the reeds L2 and L5 to form the tube-shaped double-raschel knitted fabric tube W for artificial blood vessels.

In the case of the double Denbigh tissue B, the knitting thread is moved from a needle to a needle at the time of knitting, and therefore the twist effect and the coating effects described above can be further achieved.

In the above settings, in the present embodiment, six knitting tests were conducted by using (1) three front needles FN and three back needles BN, (2) five front needles FN and five back needles BN, (3) seven front needles FN and seven back needles BN, (4) eleven front needles FN and eleven back needles BN, (5) fifteen front needles FN and fifteen back needles BN, and (6) nineteen front needles FN and nineteen back needles BN, respectively.

As a result, with the double-raschel knitted fabric tube W for artificial blood vessels being flattened, its width was (1) 1.5 mm, (2) 2.0 mm, (3) 3.0 mm, (4) 5.0 mm, (5) 6.5 mm, and (6) 8 mm.

Also, the thickness of the tube wall (tube wall thickness) was 0.1 mm to 0.3 in every case. The double-raschel knitted fabric tube W for artificial blood vessels has an outer diameter (tube outer diameter) calculated from the width described above was approximately (1) 1 mmφ, (2) 1.3 mmφ, (3) 2.0 mmφ, (4) 3.0 mmφ, (5) 4 mmφ, and (6) 5 mmφ, and the double-raschel knitted fabric tube W for artificial blood vessels having a small tube outer diameter of 1 mm to 5 mm was obtained.

### Second Embodiment

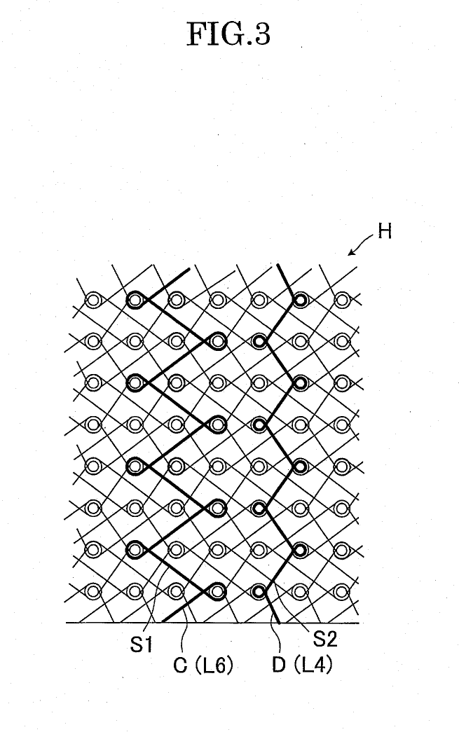

In the present embodiment, as a knitted tissue, a reverse half tissue H (1-2/1-0 is knitted by the reed L4 and 1-0/2-3 is knitted by the reed L6) depicted in FIG. 3 (which is an enlarged view of the portion P in FIG. 1, and parts are indicted by bold lines for ease of understanding) is adopted.

Other conditions are similar to those of the first embodiment.

Six knitting tests were conducted in a manner similar to that of the first embodiment. While both of the thickness and the width are increased at a level not appearing in numerical values, the results having approximately same tendencies as those of the first embodiment were obtained.

In the reverse half tissue H, in a tissue formed of a Denbigh tissue D and a code tissue C, a sinker loop 51 of the code tissue C of the code tissue C is longer by one needle than a sinker loop S2 of the Denbigh tissue D, and a dense knitted fabric can be obtained by that amount. In the case of using as the double-raschel knitted fabric tube W for artificial blood vessels, blood leakage is effectively prevented.

In the case of this reverse half tissue H, the knitting threads are moved more to a needle at the time of knitting, compared with the case of the Denbigh tissue. Therefore, the twist effect and the coating effects are achieved more.

Knitting was performed in the embodiments described above. Since a twist process is performed in addition to the fact that the sericin-coated silk thread 5 has flexibility and strength suitable for knitting, no thread breakage or fluffing of the organzine silk thread T even in a warping process of reeling the knitting thread Y to a beam or in a knitting process by the double-raschel reed R, and favorable knitting can be performed. As a completed double-raschel knitted fabric tube W for artificial blood vessels with a tube outer diameter as an aperture, a high-quality product without damage was obtained.

Also, a chemical treatment and the like were tried to provide a conventionally-known function as an artificial vessel to the double-raschel knitted fabric tube W for artificial blood vessels, and no special problem was observed.

Furthermore, even in an actual biological test, no ravel occurred at the time of suture with a living blood vessel, and the results with favorable biocompatibility were obtained.

While the present invention has been described by taking its embodiments as examples, the present invention is not restricted to the embodiments unless the gist of the present invention is changed, and various modification examples are possible.

For example, the amount of the residual sericin 13 is not necessarily within the range of 20% to 40% of the total sericin amount contained in one thread of raw silk, and can be increased or decreased according to the use situation.

Also when the organzine silk thread T is formed, the combination of twist directions and the numerical values of the number of twists for the first twist and the number of twists for the second twist can be changed, as a matter of course, according to the state or use purpose of the sericin-coated silk thread 5 for use.

Furthermore, the tissue for forming the double-raschel knitted fabric tube W for artificial blood vessels is not necessarily restricted to the double Denbigh tissue B or the reverse half tissue H.

Still further, the present invention can be used for not only an artificial blood vessel with a small diameter but also an artificial blood vessel with a large diameter.

Still further, as a matter of course, the double-raschel knitted fabric tube W for artificial blood vessels can be knitted by adding another thread material to the knitting threads Y in the present invention.

### INDUSTRIAL APPLICABILITY

In refinement of the raw silk 2 by the enzyme in the present invention, the sericin-coated silk thread 5 configured of the skin core structure fibroin 4 with a skin core structure can be formed, thereby improving knitting ability and other functionalities. The present invention can be used also for manufacturing medical materials in not only the artificial blood field but also other medical fields.

Still further, the present invention can be sufficiently used also as a silk thread material not only for medical purposes but also for clothing.

Still further, the double-raschel knitted fabric tube W for artificial blood vessels formed in a tube shape can be sufficiently used not only as an artificial blood vessel but also in a nerve system field and other medical fields.

Still further, with chemical characteristics and physical characteristics of silk it self, the double-raschel knitted fabric tube W for artificial blood vessels obtained by the present invention can be applied as a material not only in the medical field and the clothing field but also in the electronic field and the like.

### REFERENCE SIGNS LIST

1 . . . cocoon filament

11 . . . fibroin

12 . . . sericin

13 . . . residual sericin

2 . . . raw silk

3 . . . silk threads

4 . . . fibroin with a skin core structure

41 . . . core part

42 . . . skin part

5 . . . sericin-coated silk threads

A . . . contact part

B . . . double Denbigh

BN . . . back needle

C . . . code tissue

D . . . double Denbigh

FN . . . front needle

H . . . reverse half tissue

K1 . . . front knitted fabric

K2 . . . back knitted fabric

L1 to L6 . . . reed

N . . . needle

N1, N2 . . . needle loop

P . . . tissue portion of a double-raschel-knitted tube for artificial blood vessels

R1 . . . left twist direction

R2 . . . right twist direction

S1 . . . sinker loop of the code tissue

S2 . . . sinker loop of a Denbigh tissue

T . . . organzine silk thread

T1 . . . second-twist silk thread

W . . . double-raschel-knitted tube for artificial blood vessels

Y (Y1 to Y6) . . . knitting thread

1. A method of manufacturing a double-raschel-knitted tube for artificial blood vessels, the method comprising: refining raw silk configured of N cocoon filaments each formed of paired core parts formed of fibroin and a skin part surrounding the core parts and formed of sericin; separating the paired core parts with the sericin partially removed and partially left to form 2N plies of fibroin with a skin core structure to obtain a sericin-coated silk thread configured of the 2N plies of fibroin having the skin core structure; then subjecting the sericin-coated silk thread to a first twist to produce a first-twist silk thread; further combining a plurality of the first-twist silk threads together and subjecting the combined first-twist silk threads to a second twist to produce an organzine silk thread; and then further knitting a tube shape by using the organzine silk thread as a knitting thread.

2. The method of manufacturing the double-raschel-knitted tube for artificial blood vessels according to claim 1, wherein
the organzine silk thread has a number of twists for the second twist in a direction in reverse to a first twist direction of the first-twist threads, the number of twists for the first twist being smaller than a number of twists for the first twist.

3. The method of manufacturing the double-raschel-knitted tube for artificial blood vessels according to claim 1, wherein
the residual sericin is 20% to 40% of a total sericin weight contained in one thread of the raw silk.

4. The method of manufacturing the double-raschel-knitted tube for artificial blood vessels according to claim 1, wherein
the first-twist silk threads are two threads.

5. The method of manufacturing the double-raschel-knitted tube for artificial blood vessels according to claim 1, wherein
the organzine silk thread is used to knit the tube shape with a double Denbigh tissue.

6. The method of manufacturing the double-raschel-knitted tube for artificial blood vessels according to claim 1, wherein
the organzine silk thread is used to knit the tube shape with a reverse half tissue.

7. The method of manufacturing the double-raschel-knitted tube for artificial blood vessels according to claim 1, wherein
the first twist is 900 t/m to 1200 t/m, the second twist is 700 t/m to 1000 t/m, a finishing course density is 40 c/inch to 50 c/inch, and a finish wale density of 50 w/inch to 70 w/inch.

8. The method of manufacturing the double-raschel-knitted tube for artificial blood vessels according to claim 1, wherein
a tube wall thickness is 0.1 mm to 0.3 mm, and a tube outer diameter is 1 mm to 5 mm.

9. The method of manufacturing the double-raschel-knitted tube for artificial blood vessels according to claim 1, wherein
an enzyme is used to refine the sericin.

10. The method of manufacturing the double-raschel-knitted tube for artificial blood vessels according to claim 1, wherein
the residual sericin is removed after knitting.

11. A method of manufacturing a double-raschel-knitted tube for artificial blood vessels obtained by
subjecting a sericin-coated silk thread to a first twist to produce a first-twist silk thread, and
further combining a plurality of the first-twist silk threads together and subjecting the combined first-twist silk threads to a second twist to produce an organzine silk thread and knitting a tube shape by using the organzine silk thread as a knitting thread, wherein
the sericin-coated silk thread is configured of fibroin with a skin core structure, the fibroin with the skin core structure being formed by refining raw silk configured of a cocoon filament formed of paired core parts formed of fibroin and a skin part coating the core parts and formed of sericin and separating the paired core parts with the sericin partially removed and partially left.
